(19) United States
(12) Patent Application Publication
(10) Pub. No.: US 2019/0159792 A1
(43) Pub. Date: May 30, 2019

(54) Ultrasound Vessel Preparation and Restenosis Therapy

(71) Applicant: Justin Panian, San Francisco, CA (US)

(72) Inventor: Justin Panian, San Francisco, CA (US)

(21) Appl. No.: 16/027,082

(22) Filed: Jul. 3, 2018

Related U.S. Application Data
(63) Continuation of application No. 15/823,193, filed on Nov. 27, 2017, now abandoned.

(51) Int. Cl. A61B 17/22; A61M 25/10; A61B 17/3207; A61M 37/00

(52) U.S. Cl. CPC A61B 17/22012; A61M 25/104; A61M 2025/1075; A61M 25/10184; A61B 2017/22051; A61M 2025/109; A61M 37/0092; A61B 17/320725

(57) ABSTRACT

Methods and devices are disclosed for treating vascular stenosis using ultrasound vibrational energy from inside and outside of the balloon device into a treatment region of a patient's body. The vibrational energy is of a type and in an amount sufficient to remodel and alter compliance of the plaque at the treatment region and augment delivery therapeutic drugs to the treatment area.

### BACKGROUND OF THE INVENTION

### 1. Field Of The Invention

The present invention relates generally to medical devices and methods. More particularly, the present invention relates to apparatus and methods utilizing ultrasonic energy for plaque remodeling and localized delivery of therapeutic drugs within the vasculature, aortic heart valves and other body lumens,

### 2. Description Of The Related Art

The majority of current endovascular angioplasty therapies utilize dilation catheters or balloon catheters to cross a lesion, dilate the lesion and restore normal blood flow in the artery or vein. Harder lesions and particularly calcified lesions require high pressures, often 25 atm or more, to break the plaque and push it back into the vessel wall. Clinical evidence shows that such high balloon pressure can cause injury to the vessel wall which can contribute to vessel recoil, dissection, thrombus formation, and consequently cause a high restenosis rate.

Balloon angioplasty remains the first line therapy and no significant improvements have been commercialized in the last 30 years to reduce balloon dilatation pressure to minimize vessel trauma with a lower balloon pressure while maintaining the ability to satisfactorily dilate the vessel.

The growing sophistication of medical technology today includes employing local delivery of therapeutic drugs to prevent restenosis including: injection of drugs to the treatment area; delivery of drugs using drug eluting balloons, metal stents and absorbable stents. However, local delivery of drugs to the treatment area faces a serious obstacle when harder plaque or calcifications are present. Such calcification may be spread within the luminal structure of the vessel as well as in the media/intima layer of the vessel wall. While use of atherectomy devices may effectively remove/cut or minimize luminal calcifications, calcium located in the media/intima layer of the vessel wall is very difficult, if not impossible, to remove, Such calcifications present a challenge to the delivery of drugs to the vessel wall in areas where these calcifications are present, thereby limiting the efficacy of drugs to prevent endovascular restenosis.

A new approach to address vascular calcification has been proposed by Wallace (U.S. Pat. No. 9,375,223) by using ultrasound for vessel preparation and for the use of therapeutic drug in a liquid form. Although this approach has been found promising, there are several challenges that may contribute to vessel preparation using ultrasound energy and liquid drug delivery. Particularly, in less calcific and more fibrous lesions, ultrasound energy may not be effective. Also, therapeutic drug delivered in a proposed liquid form may not always be present in a portion of liquid delivered to the treatment site.

At present, there is no specific treatment for aortic stenosis and calcification. If the aortic valve becomes severely narrowed, aortic valve replacement surgery may be necessary. A balloon valvuloplasty is useful as a measure to relieve symptoms in patients who are not candidates for aortic valve replacement. However, the balloon pressure of balloon valvuloplasty rarely exceeds 5-6 atm to avoid damage to the valves, thereby limiting better clinical outcomes. Low pressure and more effective balloon valvuloplasty may improve ventricular muscle function, and people who respond to valvuloplasty with improvement in ventricular function can be expected to avoid aortic valve replacement. Thus, there is a need for a more effective aortic valvuloplasty.

Accordingly, the use of ultrasonic energy has been proposed to reduce balloon dilatation pressure and remodeling of a surrounding plaque and/or changes in compliance of calcifications to augment drug penetration into the vessel wall, as well as to increase absorption of delivered drugs delivered from balloon devices into the vessel wall of the treatment areas.

Ultrasound-based technologies are being utilized to an increasing extent, and provide the potential to improve drug administration to patients clinically. Ultrasound energy is used in transdermal drug delivery, gene delivery, and ocular drug delivery, and may be helpful in increasing vessel permeability, drug transportation to a desirable treatment area, and plaque remodeling.

### SUMMARY OF THE INVENTION

The invention provides dilatation balloon devices having a guidewire lumen in a rapid exchange fashion (RE) or an over-the-wire fashion (OTW). The balloon devices are suitable to receive a fluid that inflates the balloon. The balloon devices may have incorporated therewith a drug eluting feature on its surface or in a form of another device located on the balloon, such as for example, drug eluting stents or bioabsorbable stents with drugs. An ultrasound emitting member is extended inside the balloon device to deliver ultrasound vibrational energy from the inside of the balloon outward to the adjacent vessel/tissue.

The ultrasound emitting member comprises at least one ultrasound energy propagating metallic wire or multiple ultrasound energy propagating wires, which may be deployed inside the balloon device as well. The ultrasound propagating member may be positioned inside the guidewire lumen, outside of the guidewire lumen, or both, If desired, the ultrasound transmission emitting member may be attached to the distal end of the balloon device.

The balloon device may be formed of non-compliant material or of a compliant material. The balloon device of the present invention includes balloon material, a balloon device shaft and a guidewire lumen which are made of materials having acoustic properties suitable for stenosis composition and surrounding liquids.

The fluid to inflate the balloon device may be saline, contrast or saline deluded contrast at any suitable rate/proportion as preferred by the physician.

The present invention provides methods and devices for changing the compliance of calcific plaque, enhancing cellular absorption of a drug or other substance to the treatment area located within the patient endovascular system or outside of the patient endovascular system including aortic valves.

The present invention also includes balloon devices having a drug coated balloon, a balloon with drug eluting stents or balloons with absorbable stents having drugs, as well as a balloon with micro-holes around its outside surface. A liquid drug may be pressurized from inside the balloon into the surrounding vessel wall in conjunction with the delivery of ultrasound vibrational energy. The application of such vibrational energy of the present invention to the target treatment region may change the plaque compliance and increases cellular absorption on the order of 5 to 500 percent or more for biological reporters such as luciferase gene beta-galactosidase and for drugs such as heparin, probucol, the family of anti-cancer vectors, liposome-complexed plasmid DNA, adeno-associated virus, vascular endothelial growth factors, and naked DNA relative to their uptake in the absence of the vibrational energy.

The present invention is useful for delivering a wide variety of drugs suitable to improve restenosis.

In one embodiment, ultrasound vibrational energy is delivered from within the drug eluting balloon and dispersed through the wall of the balloon device to the surrounding treatment area, while at the same time, the therapeutic drug is activated by ultrasound waves and carries towards the treatment area.

Ultrasound surface induced waves and spherical waves propagate radially from the ultrasound emitting member through the balloon device and facilitate therapeutic drug movement into the treatment area.

Also, the balloon device may include a balloon with a drug coated metallic or bioabsorbable stent loaded on the surface of the balloon. Upon inflation of the balloon, the drug coated stent is expanded against the treatment area, while ultrasound vibrational energy is activated. Spherical waves propagate radially from the ultrasound emitting member to the stent surface, thus facilitating therapeutic drug movement into the treatment area.

In another embodiment, the balloon device has a balloon fabricated from a porous elastomeric material with a plurality of voids. The voids are loaded with drugs in various formulations. Upon inflation of the balloon, the voids are stretched to cause the drug to be expelled from the voids and into the bodily lumen or adjacent tissue. Activation of ultrasound spherical waves will further vibrate or shake the surface of the balloon and facilitate drug penetration into the treatment area.

Other embodiments of the present invention include systemic delivery of bubbles with therapeutic agents while delivering ultrasound energy from within the balloon device.

In another embodiment, a balloon device may be configured to be placed adjacent leaflets of a valve, the balloon device having a first portion to be positioned adjacent one side of the leaflets and a second portion to be positioned adjacent an opposite side of the leaflets. A therapeutic drug is delivered to the leaflets in conjunction with delivery of ultrasound vibrational energy to further facilitate drug movement to the treatment area.

Other embodiments of the present invention may include additional diagnostic, measurement, or monitoring components or capabilities. For example, the device for emitting vibrational energy to the target region may be adapted to detect the acoustic impedance of the target tissue in opposition with the vibrational device, thus enabling an operator to determine procedural length to compensate for acoustic impedance mismatches.

Various perfusion catheters are known in the art, and other catheters employ ultrasound energy in conjunction with drug delivery. However, the prior art is silent about combination therapy including vessel preparation with ultrasound energy and/or balloon angioplasty, drug delivery using perfusion balloons to change plaque compliance, increasing drug cellular absorption, and safe delivery of drugs to the treatment area while delivering ultrasound energy using one device.

“Cellular Absorption” means that a significant proportion of the total amount of drug delivered to the treatment site is absorbed or otherwise uptaken into cellular space between cells. The nature of the cells may vary depending on the target site. The cells may include but are not limited to vascular cells. These cells may be muscle cells, fat cells or epithelial cells, and/or other cells which line the interior or exterior of target organs, or brain cells protected by the blood/brain barrier, or organ cells in general. The cells may also be specific organ cells of a target organ.

“Plaque/Atherosclerotic Plaque” means fibrous tissue, fibro-fatty tissue, necrotic core, calcified tissue, dense calcium and a combination thereof.

“Change of Plaque Compliance” means that additional changes to the plaque structure are induced, including but not limited to, cutting, ablating, shaking, creating micro-channels, creating cracks, and a combination thereof.

“Plaque Remodeling” means an enlargement in de novo atherosclerotic plaque or in restenosis to prevent or reduce additional plaque formation or intimal hyperplasia.

“Vessel Preparation” means change of vessel plaque compliance and making the vessel primed and ready for a therapeutic drug application.

### BRIEF DESCRIPTION OF THE DRAWINGS

Objects and advantages of the present invention and methods and devices will become clear to those skilled in the art from the following detailed description, wherein only the preferred embodiments are shown and described, simply by way of illustration of the best mode contemplated of carrying out the invention. As will be realized, the invention is capable of other and different embodiments and methods of construction, and its several details are capable of modification in various obvious respects, all without departing from the invention. Accordingly, the drawing and description are to be regarded as illustrative in nature, and not as restrictive.

FIG. 1 shows an overall view of an inflated balloon device according to the present invention.

FIG. 2 illustrates the balloon device of FIG. 1 introduced over a guidewire into the treatment area and prior to balloon inflation.

FIG. 3 illustrates an ultrasound device according to the present invention.

FIG. 4 shows the ultrasound device of FIG. 3 positioned at the treatment site and activated.

FIG. 5 illustrates the balloon device of FIG. 2 inflated with the guidewire removed and with an ultrasound device of FIG. 3 positioned inside the balloon device.

FIG. 6 shows a drug eluting balloon at the treatment site, and ultrasound energy delivered to change the compliance at the treatment site, and to facilitate drug penetration to the vessel wall.

FIG. 7 demonstrates the ultrasound device of FIG. 3 positioned at the treatment site and activated, after the drug eluting balloon of FIG. 6 has been removed from the treatment site.

FIG. 8 illustrates a balloon device with a balloon having micro-holes, and ultrasound emitting member located inside the guidewire lumen and activated inside the balloon, with therapeutic drug infused from within the balloon device to the treatment area.

FIG. 9 shows a balloon device without a guidewire lumen and with an ultrasound emitting member extended inside the balloon.

FIG. 10 shows a balloon device with a guidewire lumen and with an ultrasound emitting member comprising multiple wires extended inside the balloon device and outside the guidewire lumen.

### DESCRIPTION OF THE PREFERRED EMBODIMENTS

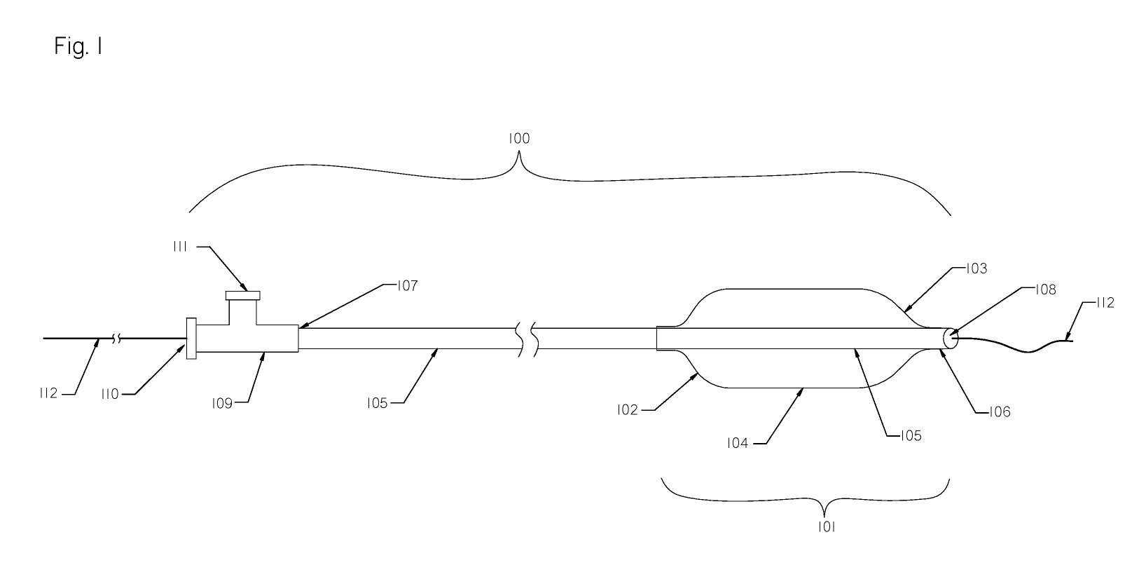

FIG. 1 shows the balloon device 100 which generally comprises a dilatation balloon 101 shown in an inflated stage having the proximal neck portion 102, a distal neck portion 103, and an intermediate dilatation portion 104. The balloon 101 is mounted on a balloon device shaft 105 having a distal end 106 and a proximal end 107. A separate guidewire lumen 108 is extended within and along the length of the balloon device shaft 105. The proximal end 107 of the balloon device shaft 105 and the proximal end of the guidewire lumen (not shown) are both attached to a Y-connector 109. The Y-connector 109 has a guidewire port 110 and a balloon inflation port 111. The balloon inflation port 111 provides access for inflating the balloon 101, while the port guidewire 110 provides access for the guidewire 112 and other devices (as needed) to be introduced through the balloon device 100,

The balloon 101 of the balloon device 100 may be formed of non-compliant material or a compliant material. The balloon device 100 may have a guidewire lumen in a rapid exchange fashion (RE) or an over-the-wire fashion (OTW). The balloon device 100 is adapted to receive a fluid that inflates the balloon 101.

The balloon 101 preferably comprises a relative non-elastic material, such as medium density polyethylene, linear low-density polyethylene, polyethylene teraphthalate, nylon, polyester, or any of a variety of other medical grade polymers known for this use in the catheter balloon art Preferably, the geometry and balloon materials can withstand an internal pressure of at least 10 atmospheres without any leakage or rupture. The fluid to inflate the balloon 101 may be contrast or saline deluded x-ray contrast at any suitable rate as preferred by the physician.

The balloon 101 may include a conventional balloon or a drug eluting balloon (DEB) with a therapeutic drug incorporated on the surface of the dilation balloon 101 (not shown), Also, a bare metal stent (BMS—not shown), a drug eluting stent (DES—not shown) or a balloon with a bioabsorbable stent (BAS—not shown) may be incorporated or positioned on the dilatation balloon 101.

The balloon devices as described above such as; DEB, DES, BMS, BAS are well known in the art, therefore, no detailed descriptions are provided herein. However, the materials for the balloon device 100, specifically the balloon 101, balloon device shaft 105 and guidewire lumen 108, all are within the field of clinical ultrasound energy propagation, and preferably should exhibit acoustic characteristics that enhance ultrasound energy propagation in a clinical setting.

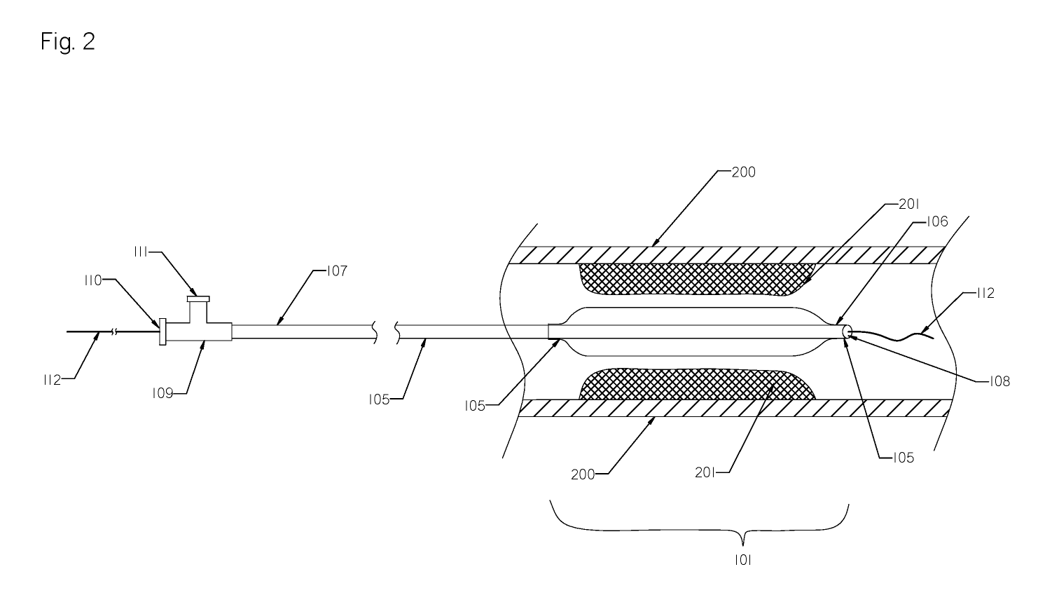

FIG. 2 shows the balloon device 100 of FIG. 1 in an inflated stage positioned inside the vessel wall 200 and within the treatment area 201. The treatment area 201 comprises atherosclerotic plaque that may include clots, fibrous tissue, fibro-fatty tissue, necrotic core, calcified tissue, dense calcium, or a combination thereof. The balloon 101 of the device 100 is introduced to the treatment area 201 in a deflated state over the guidewire 112, and once within the treatment area 201, is inflated accordingly.

The treatment area 201 may be pre-treated prior to the introduction of the balloon device 100 by performing additional angioplasty at the treatment area 201, including one of the following: balloon angioplasty, laser angioplasty, lithoplasty, atherectomy, thrombectomy, ultrasound angioplasty, cutting balloon plasty or any combination thereof.

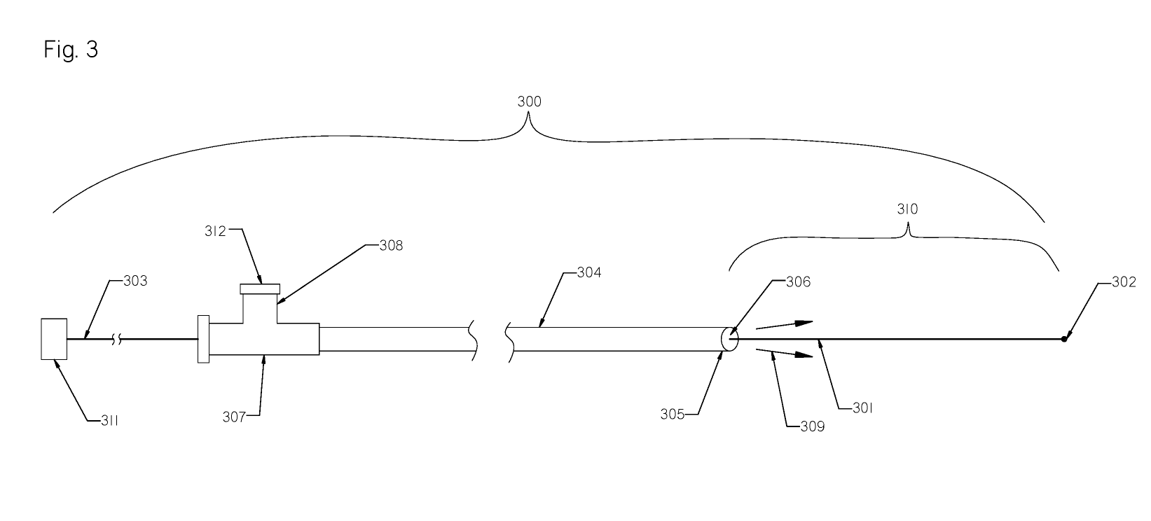

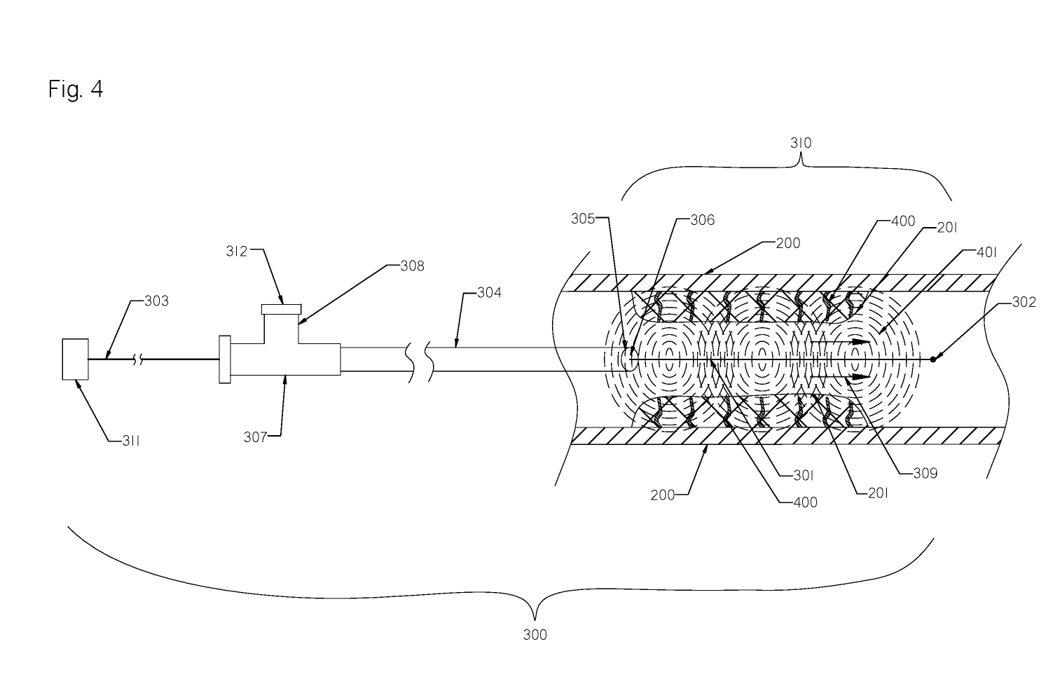

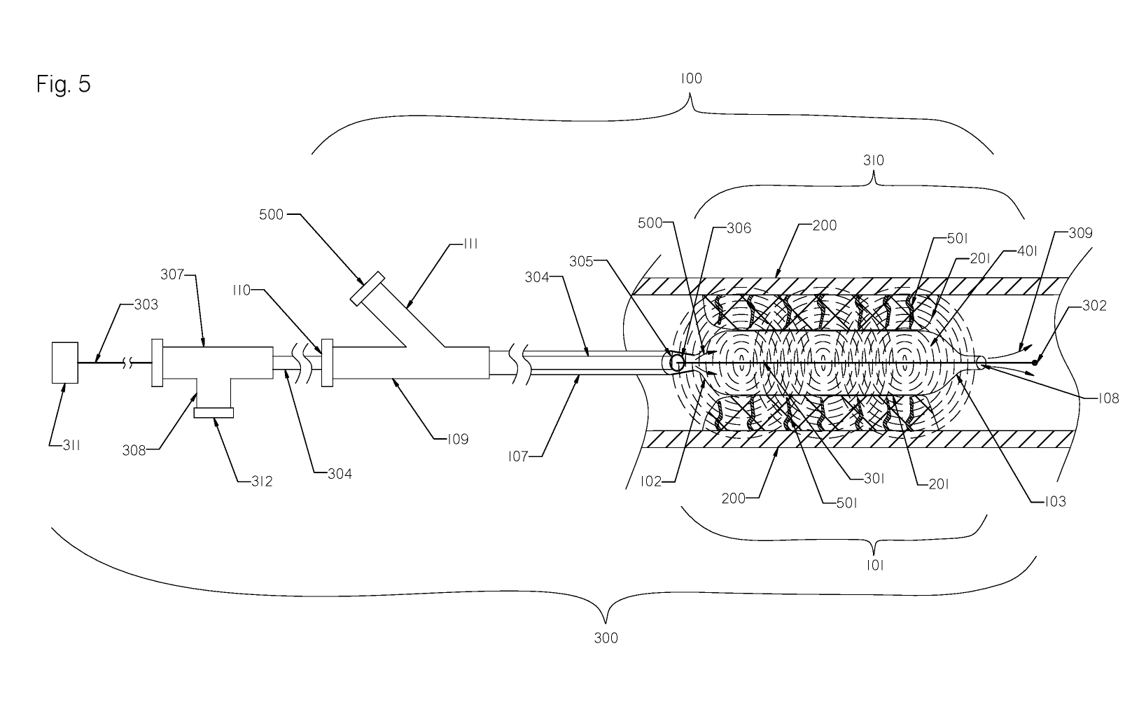

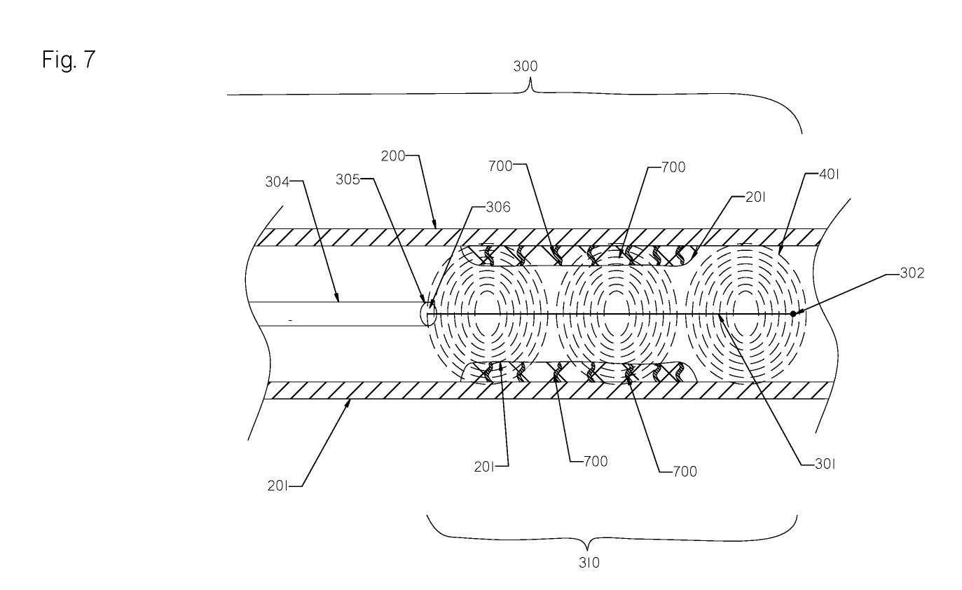

FIG. 3 shows an ultrasound device 300 that may be used alone as shown in FIGS. 4 and 7, or in conjunction with the balloon device 100 as shown in FIGS. 5, 6 and 8. The ultrasound device 300 comprises an ultrasound emitting member 301 having a distal end 302 and a proximal end 303. The ultrasound emitting member 301 is extended at least partially inside a catheter shaft 304 having a distal end 305 and an inner lumen 306, and is proximally attached to a Y-connector 307. The Y-connector 307 includes a port 308 having an entry 312 for the delivery of irrigation 309 along the inner lumen 306 of the catheter shaft 304. The proximal end 303 of the ultrasound emitting member 301 is attached to an ultrasound energy transducer 311 such that ultrasound vibrations propagate from the energy transducer 311 into the proximal end 303 of the ultrasound emitting member 301, and through the entire length of the ultrasound emitting member 301 to its distal end 302.

During the delivery of ultrasound energy, a saline irrigation 309 is delivered through the entry 312 of the port 308 and along the ultrasound catheter shaft 304 to provide cooling of the ultrasound emitting member 301, When ultrasound energy is delivered from the energy transducer 311 into the proximal end 303 of the emitting member 301, it propagates through the emitting member 301 to its distal end 302. The irrigation 309 is delivered through the entry 312 of the port 308 of the Y-connector 307 along the emitting member 301 and exits at the end 305 of the inner lumen 306. Since the irrigation 309 is delivered under pressure into the entry 312 of the port 308, it continues to flow along the distal portion 310 of the ultrasound emitting member 101, and surrounding the distal portion 310 of the ultrasound emitting member 301, The irrigation 309 may be provided into the entry 312 of the port 308 by any suitable means including a syringe, pumps or pressure bags. The irrigation 309 surrounding the distal portion 310 of the ultrasound emitting member 301 will create a liquid media to further propagate ultrasound energy radially into the treatment area 201 and the vessel wail 200. The irrigation medium may comprise saline (also known as saline solution), a mixture of sodium chloride in water that has several uses in medicine such as cleaning wounds, helping to remove contact lenses and with dry eyes, and commonly used to treat dehydration by systemic injections, and often used to dilute other medications to be given by injection,

The ultrasound emitting member 301 may comprise one of the following configurations: distally tapered, multi-segments having smaller and larger cross sections, or a combination of both. The very distal end 302 of the ultrasound emitting member 301 may comprise a rounded end, fused ball or attached tip (not shown) to provide a less traumatic termination for the distal end 302 of the ultrasound emitting member 301, A fused ball or attached tip may provide better angiographic visibility to safely position and reposition the distal end 302 of the emitting member 301 within and outside of the treatment area 201. The ultrasound emitting member 301 is preferably made of metal or metal alloys, and has any cross-sectional configuration as applicable for clinical use, including but not limited to rounded, square, oval or others. The emitting member 301 may include single or multiple wires distally or along the entire length.

FIG. 4 illustrates the ultrasound device 300 of FIG. 3 positioned at the treatment site 201 inside the vessel wall 200 with ultrasound energy delivered from the energy transducer 311 into the proximal end 303 of the ultrasound emitting member 301 and then to the distal portion 310 and the distal end 302. Such vessel preparation, or the changing of vessel plaque compliance using the ultrasound energy within the treatment area, may be helpful for remodeling of the treatment area 201 by reducing additional plaque formation, inducing micro-cracks and microchannel within the plaque, and consequently achieving better clinical outcomes,

Ultrasound energy delivered from the energy transducer 311 and along the ultrasound transmitting member 301 may have a frequency between 100 Hz and 10 MHz, preferably 17-100 kHz, and may be in continuous mode of operation, pulse mode of operation, or a combination of both. It is important to mention that attenuation of the ultrasound waves in the body or materials is increased as frequency is increased due to greater absorption, so kHz level frequency propagation may be more desirable for therapeutic uses.

Ultrasound waves propagate by causing local oscillatory motion of particles through the medium through which they are traveling, either in solids or liquids. As the sound wave displaces particles at a given location, the local density and pressure of the medium increases or decreases depending on whether that location is in a rarefaction (low pressure) or compression (high pressure) cycle of the wave, causing acoustic cavitation which is generally accepted as the main contributor in the use of ultrasound energy.

It is important to stress that high-frequency ultrasound will generate bubble populations having smaller radii than low-frequency ultrasound. For example, the linear resonant bubble radius of air bubbles in water, which is assumed to be incompressible and inviscid, for ultrasound at 20 kHz is 150 μm, while at 3 MHz it is only 1 μm. Thus, this shows frequency implications on the level of enhancement that can be achieved at lower ultrasound frequencies.

In addition to cavitation, there are several other mechanisms which may have therapeutic roles such as convection, thermal effects, and mechanical or radiation pressure effects, among others.

Ultrasound energy propagates from the energy transducer 311 and through the ultrasound emitting member 301 in longitudinal waves. From the distal portion 310 of the ultrasound emitting member 301, ultrasound energy propagates radially in the form of surface waves that induce spherical waves 401 radially to the treatment area 201. These waves have characteristics suitable for use in medical therapies, meaning that a healthy tissue (elastic tissue) exposed to these ultrasound waves will not suffer any injuries, Other ultrasound waves may include shear (transverse) waves, Lamb, Love, Stoneley or Sezawa, but do not play any therapeutic role. Surface waves are mechanical waves that propagate along the interface between differing media. If the ultrasound transmission member 301 is submerged in liquid or surrounded by liquids such as irrigation fluid 309, surface waves (not shown) will be created around the ultrasound emitting member 301 within the surrounding irrigation fluid 309. While surface waves propagate radially into neighboring liquid irrigation, they create a cavitational effect along its encircling radial path, emitting bubbles and pressure waves radiating outwardly from the bubble creating spherical waves 401. Such surface waves induced spherical waves 401 have a great potential to shock, shake or disturb diseased tissue and calcific plaque, as shown by arrow 400, thereby changing compliance of the treatment area 201 and facilitating drug delivery through plague 201 to the vessel wall 200,

FIG. 5 shows the balloon device 100 of FIG. 2 positioned within the treatment area 201 inside the vessel 200 with the guidewire 112 removed, and with the ultrasound device 300 of FIG. 3 extended through the port 110 of the balloon device 100 into the guidewire lumen 108. The distal end 302 of the ultrasound emitting member 301 is positioned outside of the distal end 106 of the balloon device 100. The distal end 305 of the ultrasound catheter shaft 304 is positioned within the guidewire lumen 108 prior to the proximal neck 102 of the balloon 101. The proximal end 303 of the ultrasound transmitting member is attached to ultrasound energy transducer 311. The ultrasound emitting member 301 is positioned inside the balloon 101 of the balloon device 100 to deliver ultrasound vibrational energy from the inside of the balloon 101 outwardly to the adjacent vessel/tissue.

The balloon 101 of the balloon device 100 is inflated against the treatment area 201 within the vessel 200 using conventional inflation devices (not shown) filling the interior of the balloon 101 with a mixture of saline and contrast 500 delivered through the port 111. Ultrasound energy propagates from the ultrasound energy transducer 311 into the proximal end 303 of the ultrasound emitting member 301 towards the distal portion 310 of the ultrasound emitting member 301 and the distal end 302. The distal portion 310 of the ultrasound emitting member 301 is located within the distal portion of the guidewire lumen 105 and within the balloon 101 of the balloon device 100.

During delivery of ultrasound energy into the emitting member 301, a saline irrigation 309 is supplied along the ultrasound catheter shaft 304 to provide cooling of the ultrasound emitting member 301. A saline irrigation 309 enters through the entry 312 of the proximal port 308 of the Y-connector 307 and exits from the inner lumen 306 at the distal end 305 of the ultrasound catheter shaft 304 as shown by arrows 309. Since the distal end 305 of the ultrasound catheter shaft 304 is positioned within the guidewire lumen 108 proximal to the balloon 101, saline irrigant 309 flows around the proximal portion 310 of the ultrasound emitting member 301 inside the guidewire lumen 108 and exits outside at the distal end 106 of the balloon device 100.

In addition to cooling the ultrasound emitting member 301, saline irrigation 309 provides an acoustic coupling between the distal portion 310 of the ultrasound emitting member 301 and the wall of the guidewire lumen 108 and the balloon device shaft 105.

Ultrasound longitudinal waves delivered from the energy transducer 311 and propagating into the proximal end 303 of the ultrasound emitting member 301 are discharged by propagating radially from the distal portion 310 of the ultrasound emitting member 301. While propagating radially from the distal portion 310 of the ultrasound emitting member, ultrasound spherical waves reflect from the boundary between different types of materials and tissue. In the present invention, spherical waves 401 propagate from the distal portion 310 of the ultrasound emitting member 301 through irrigation 309 surrounding the ultrasound emitting member 301 inside the guidewire lumen, through the guidewire lumen 108 material, through saline/contrast mixture 500 filling the balloon catheter 101, through the balloon catheter 101 material, through plaque at the treatment area 201, and then to the vessel wall 200. Ease of propagation of the ultrasound energy through these materials and the balloon device components depends on the acoustic property of the materials, called acoustic impedance.

Acoustic impedance is a physical property of materials or tissue which describe the amount of resistance an ultrasound beam encounters as it passes through a tissue or materials and is defined as:

Acoustic Impedance=Density of Material X Speed of Sound in the tissue/material, The SI unit for acoustic impedance is the Rayl, and 1Rayl=1 kg/m²s.

The greater the difference between the acoustic impedances of the two materials at a boundary in the body, the greater the amount of reflection and the lower the ability to propagate ultrasound energy. Two materials with the same acoustic impedance would give no reflection (or refraction) while two materials with different values would result in unwanted reflections and produce significant ultrasound energy loses.

Matching acoustic impedance of different material components and tissue is very important in delivering ultrasound energy inside the body or inside diseased vessels, and faces several challenges, First, the tissue at the treatment area is already present there, and may range from a clot, soft tissue to a solid calcium. In addition, blood may also be present at the treatment area. Thus, no changes, alteration or modifications are easy or even possible. Second, the structure of a device or system delivering ultrasound energy comprises several different materials and components. As described above, all the components of the balloon device 100, the ultrasound device 300, as well as saline irrigation 309 and saline/contrast mixture 500 inflating the balloon 100 exhibit different acoustic impedances.

While it is difficult or impossible to match acoustic impedance of these materials or components, using balloon device materials that exhibit similar or comparable acoustic impedance may be beneficial in improving ultrasound propagation from the ultrasound emitting member 301 to the vessel wall 200 through saline irrigation 309, the material of the guidewire lumen 108, the material of the balloon device shaft 105. saline/contrast 500 inside the balloon 100, the material of the balloon 101, and the plaque at the treatment area 201 to vessel wall 200.

It is desirable that all materials for the balloon device 100, liquids, blood and tissue as listed above exhibit similar acoustic impedance to assure the best acoustic environment for ultrasound wave propagation. For example, acoustic impedance in saline is 1.48×10⁶ Rayl; in blood is 1.65×10⁶ Rayl, in soft tissue is 1.50×10⁶ Rayl, and in calcifications is 5.86×10⁶Rayl. It is essential to provide all balloon device components and materials that do not impede ultrasound wave propagation, so the amount of vibrational energy is sufficient to be delivered through the balloon device 100 through the treatment area 201 to the vessel wall 200. Accordingly, all these materials should preferably exhibit acoustic impedance of less than 6.0×10⁶ Rayl, and not exceeding the acoustic impedance of calcium, which is the higher acoustic impedance of all materials and components present at the treatment site, including all components of the balloon device 100, the treated plaque 201, surrounding blood, saline 309 or saline/contrast mixture 400, and the vessel wall 200.

Ultrasound energy from the emitting member 301 propagates through the balloon device 100 into the treatment area 201 and towards the vessel wall 200 in the form of spherical waves as shown by arrows 401. When the ultrasound spherical waves 401 reach the treatment area 201, a local acoustic cavitation caused by these waves will change plaque compliance by creating micro-channels/cracks 501 within the harder plaque and calcifications, while in a softer plaque these waves will not make changes, and in the vessel wall will induce vasodilatation (not shown). Changing plaque compliance may be helpful in reducing dilatation pressure of the balloon 101 to achieve a nominal or original vessel dilatation. It is also known from the prior art that a lower dilatation pressure may induce less trauma into the treatment area, and such plaque remodeling by enlargement in de novo atherosclerotic plaque or in restenosis may prevent or reduce additional plaque formation or intimal hyperplasia. Inducing vasodilatation in the vessel 200 may increase vessel permeability and increase therapeutic drug absorption.

Furthermore, a systemic or local delivery of micro bubbles with therapeutic agents to the treatment area 201 while delivering ultrasound energy from within the balloon device 100 may expand the remodeling of the treatment area 201, and increase drug uptake in the vessel 200.

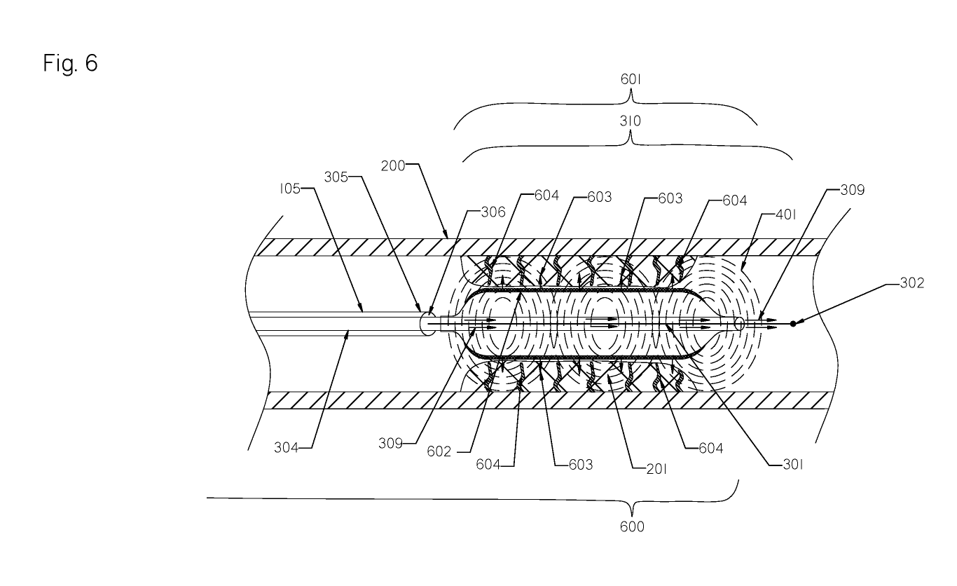

FIG. 6 shows the balloon device 600 having the drug eluting balloon 601 with a therapeutic drug 602 provided outwardly on the surface of balloon 601, and positioned at the treatment area 201 within the vessel 200. Ultrasound energy is activated and delivered from the distal portion 310 of the ultrasound emitting member 301. Ultrasound energy propagates out of the distal portion 310 of the ultrasound emitting member 301 during inflation of the balloon 601, and therapeutic drug 602 is delivered to the treatment site 201 as shown by arrows 603. Therapeutic drug 602 may penetrate from the balloon 601 surface through micro-cracks/channels 604 into the treatment area 201 towards the vessel wall 200. Ultrasound spherical waves 401 propagate from the distal portion 310 of the ultrasound emitting member 301 and serve as an additional vehicle to facilitate and improve the movement of therapeutic drug 602 from the balloon 601 through the treatment site 201 and into the vessel wall 200. Ultrasound spherical waves 401 through its cavitational effects are designated to change compliance of the treatment area 201 and to facilitate advancement of the therapeutic drug 602 located on the surface of the balloon 60, and penetration of the drug 602 through the treatment area 201 (as shown by arrows 603) and to the vessel wall 200 to further reduce restenosis.

A considerable proportion of the total amount of therapeutic drug 602 delivered to the treatment site 201 is expected to be activated by local ultrasound cavitational effects created by spherical waves 401 and absorbed or otherwise moved or uptaken into cellular space between cells of the vessel 200 while ultrasound energy is delivered from ultrasound emitting member 301. This improves uptake of the therapeutic drug in the vessel 200 and improves clinical outcomes.

The present invention will be useful for delivering a wide variety of drugs and other substances to target tissue sites. The substances will usually have a pharmological or biological effect and range from those generally classified as small molecule drugs (usually below 2 kD, more usually below 1 kD), such as hormones, peptides, proteins, nucleic acids, carbohydrates, and the like. to those generally classified as large molecule drugs (usually above 200 kD) such as complete strands of DNA. The present invention will be particularly effective in delivering macromolecules such as biologically active proteins and nucleic acids, For delivery to the muscles in general, or the myocardium in particular, useful substances may enhance angiogenesis stimulators, such as vascular endothelial growth factor (VEGF) and basic fibroblast growth factor (BFGF). Other useful substances may include endothelial nitric oxide synthase (eNOS) for inhibiting restenosis, and brain naturatic peptides, and beta-adrenogenic receptors for preventing congestive heart failure. Ultrasound in combination with DNA-based vaccines would enhance protein expression by improving the humoral and cellular immune response.

Therapeutic drugs within the scope of the present invention include but are not limited to plant alkaloids, which bind to microtubular proteins thereby inhibiting microtubule assembly, Such alkaloids include Etoposide, Paclitaxel (Taxol), Treniposide, Vinblastine (Velban, Velsar, Alkaban), Vincristine (Oncovin, Vincasar, Leurocristine) and Vindesine (Eldisine).

Other intervention may be performed at the treatment area 201 before or after the delivery of ultrasound enhanced therapeutic drug 602 from the drug eluting balloon 601 to the vessel wall 200, including but not limited to balloon angioplasty, atherectomy, thrombectomy or combination or other.

FIG. 7 shows the ultrasound emitting member 301 positioned within the treatment site 201 inside the vessel 200 after dilatation by the balloon device 100 and the delivery of ultrasound energy as in FIG. 6. The ultrasound emitting member 301 is extended beyond the distal end 106 of the balloon device 100 and positioned within the previously treated area 201. The application of ultrasound energy in a such manner will further enhance the delivery of residual therapeutic drug 700 previously delivered from the surface of the balloon 600 to the vessel wall 200, and further reduce intimal hyperplasia and minimize restenosis. Such additional application of ultrasound energy to the treatment area to increase drug penetration and permeability into the vessel wall is performed before the residual therapeutic drug 700 left at the treatment area is washed out by blood flow. Thus, such application of ultrasound should be performed within less than 30 minutes from the delivery of therapeutic drug using the drug eluting balloon 600 as in FIG. 6.

Alternatively, the ultrasound emitting member 301 may be used in the same manner prior to placement of the balloon device 100 with the drug eluting balloon 600 at the treatment area 200 to change plaque compliance at the treatment area 201 as shown in FIG. 4. Such application of ultrasound energy at the treatment site may reduce pressure by the drug eluting balloon 600 during the delivery of therapeutic drug 601 and enhance delivery of therapeutic drugs to the vessel 200.

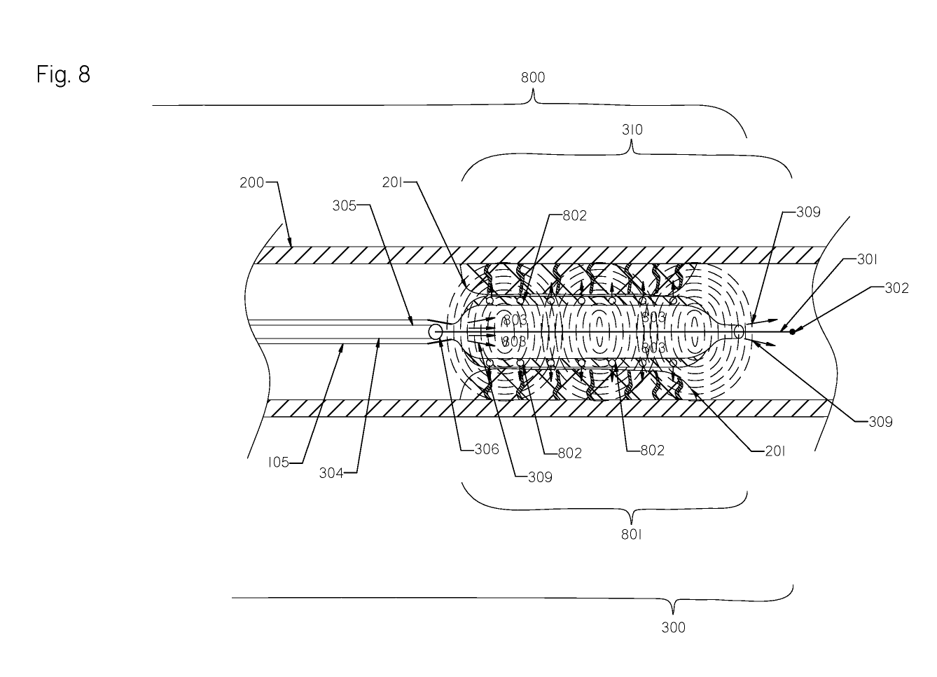

FIG. 8 demonstrates a balloon device 800 having a balloon 801 with micro-holes 802 located radially around the surface of the balloon 801. The balloon 801 is positioned at the treatment site 201 inside the vessel wall 200. The ultrasound emitting member 301 is located inside the guidewire lumen 108 of the balloon device 800, and ultrasound energy is activated. The balloon 801 is inflated and filled with a therapeutic drug in a liquid form 803, which under inflation pressure is forced through micro delivery holes 802 in the wall of the balloon 801 and into the treatment area 201 and towards the vessel 200. Therapeutic drug 803 may be delivered undiluted or in a diluted form, in mixture with saline, contrast, blood or combinations thereof.

Ultrasound energy delivered from the distal portion 310 of the ultrasound emitting member 301 which propagates across the liquid therapeutic drug 803 inside and outside of the balloon 801 further shake, agitate and facilitate the penetration of therapeutic drug 803 through the treatment site 201 towards the vessel wall 200.

The balloon 801 may be provided with a plurality of delivery holes 802 disposed radially symmetrically about the outer periphery of the delivery balloon 801, or may be limited to only portions of the exterior surface of the delivery balloon 801, depending upon the desired drug delivery pattern. For example, the delivery holes 802 can be positioned along a single line extending axially along the balloon 801, or on one hemisphere of balloon 801. Alternatively, delivery holes 802 can extend for less than the entire length of the balloon 801.

The balloon 801 may alternatively comprise a material which is inherently permeable, without the provision of discrete delivery holes 802. For example, woven or braided filaments or fabrics can be used. F or relatively low delivery rate applications, fluid permeable membranes can also be used.

Therapeutic drug is delivered from the balloon 801 under pressure into the treatment site 201 and the vessel 200. To avoid any potential trauma to the vessel 200, the injection pressure of the therapeutic drug 803 into the vessel wall 200, or pressure created on the vessel wall 200, should not exceed 5 atm.

Ultrasound energy may be delivered to the treatment site 201 prior to placement of the balloon device 800 at the treatment site 201 or after the placement of the balloon device 800 at the treatment site 201, as shown in FIGS. 4 & 7.

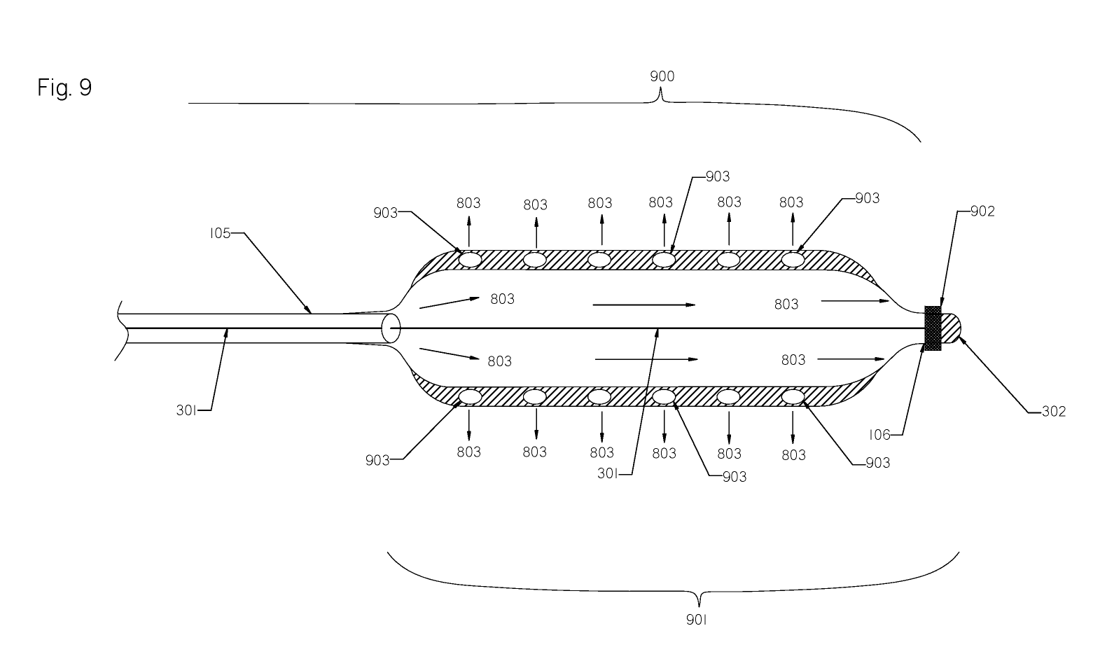

The ultrasound emitting member 301 may be extended and permanently located inside the balloon 901 with micro holes 903 of the balloon device 900. See FIG. 9. The balloon device 900 has no guidewire lumen within the balloon 901 as shown in FIG, 9. The ultrasound emitting member 301 is positioned inside the balloon 901 with its distal end 302 attached to the distal end 106 of the balloon device 900 at the attachment area 902. When the ultrasound emitting member 301 is activated and ultrasound energy propagates from the ultrasound emitting member 301, ultrasound waves agitate the therapeutic drug 803 within the balloon 901, facilitating drug penetration through the micro-holes 903 into the treatment area surrounding the balloon 901 (not shown) and ultimately add to prevention or reduction of additional plaque formation or creation of intimal hyperplasia.

In another alternative option, the balloon device 1000 comprises a balloon 1001 with micro-holes 1002 and has an ultrasound emitting member 1003 made of two wires. The emitting wires 1003 are extended inside the balloon 1001 and around the shaft 1004 and guidewire lumen 1005, and attached to the distal end 1006 of the balloon device 1000. Therapeutic drug 1007 is delivered from within the balloon 1001 to outside of the balloon 1001 via micro holes 1002 and to a treatment area (not shown). Such a configuration overcomes challenges related to the need for ultrasound energy to propagate through the balloon device shaft 1004 and the guidewire lumen 1005 as shown in FIGS. 5, 6 and 8. Thus, ultrasound energy propagates from the emitting wires 1003 directly into the therapeutic drug 1007 inside and outside of the balloon 1001. Optionally, the ultrasound emitting member can be provided in the form of as many multiple individual wires (i.e., more than two) as desired.

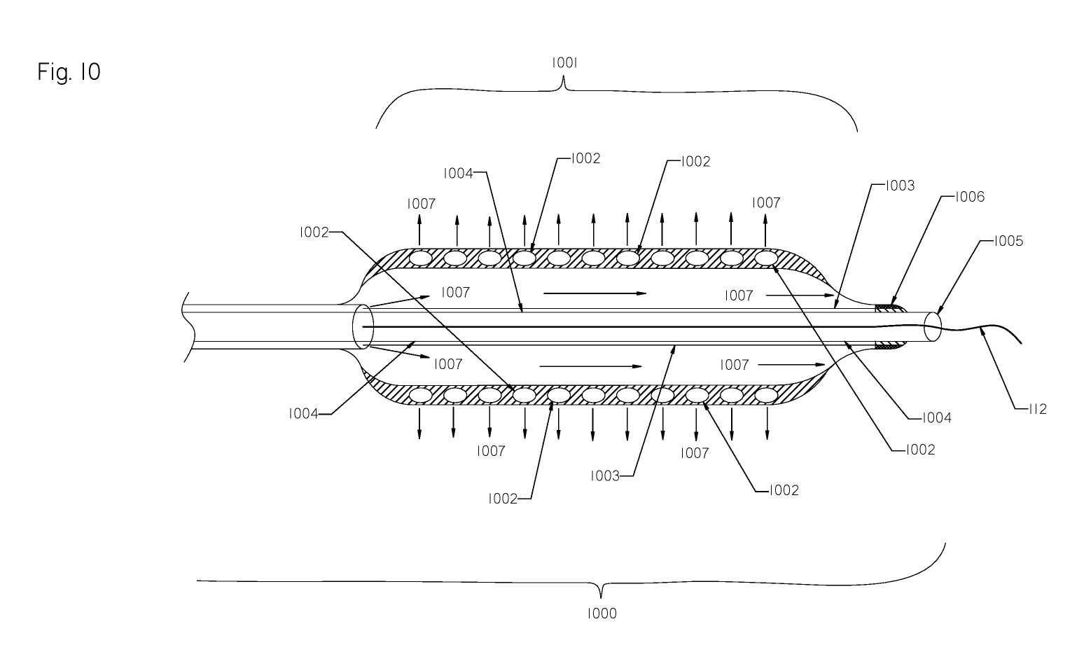

The method of placing the ultrasound emitting member or wires inside the balloon device and affixing them to the distal end of the balloon as shown in FIGS. 9 and 10 are not limited to balloons with micro-holes, but can also be utilized for any conventional balloons, drug eluting balloons and balloons with stent.

Treatment of aortic stenosis and calcifications continue to be a clinical challenge which often requires aortic valve replacement surgery. Balloon valvuloplasty is useful as a measure to relieve symptoms, but the low pressure of balloon valvuloplasty rarely exceeds 5-6 atm to avoid valve damage, thus limiting better clinical outcomes. The balloon device 100 and the ultrasound device 300 may be helpful to remodel aortic valves and facilitate a more effective balloon valvuloplasty, thus improving ventricular muscle function. People who respond to valvuloplasty with improvement in ventricular function can be expected to avoid aortic valve replacement.

Additional diagnostic, measurement, or monitoring components or capabilities may be helpful while using the methods and devices of the present invention for restenosis therapy. For example, the device for emitting vibrational energy to the target region may be adapted to detect the acoustic impedance of the target tissue in opposition with the ultrasound emitting member 301, thus enabling an operator to determine procedural length to compensate for acoustic impedance mismatches.

The methods and devices for restenosis therapy described in this specification are not only limited to the described embodiments. Any combination of methods and devices of this invention can be selected as appropriate for clinical application.

The methods of the present invention using ultrasound energy to modify plaque and enhancing cellular drug absorption into the vessel wall at the treatment area may include, but are not limited to, arteries, veins or aortic valves.

Some scientific and theoretical considerations have been introduced for assessing how these therapeutic methods are effective; these considerations have been provided only for providing an understanding of the invention only, and have no relevance to or bearing on claims made to this invention

The above described embodiments of the invention are merely descriptive of its principles and are not to be considered limiting, Further modifications of the invention herein disclosed will occur to those skilled in the respective arts and all such modifications are deemed to be within the scope of the invention as defined by the following claims

1-16. (canceled)

17. A medical device comprising:
a balloon catheter having a guidewire lumen and a balloon;
an ultrasound energy transducer located outside the balloon catheter;
an ultrasound emitting member having a proximal portion attached to the ultrasound energy transducer, and a distal portion extending longitudinally through the guidewire lumen, wherein the ultrasound emitting member delivers ultrasound energy from within the guidewire lumen and across the balloon when the balloon is inflated;
wherein the guidewire lumen and balloon material comprise polymers having acoustic impedance less than 6.0×10⁶ Rayl, wherein the ultrasound energy has a frequency range of 1 kHz-1 MHz, and wherein the ultrasound emitting member is freely positioned within the guidewire lumen and irrigation is provided inside the guidewire lumen during ultrasound delivery.

18. The device of claim 17, wherein the balloon is inflated with a radiopaque liquid and wherein the balloon comprises one of the following configurations: a balloon with micro-holes, a drug eluting balloon, a balloon with a conventional stent, a balloon with drug eluting stent, or a balloon with a bioabsorbable stent.

19. The device of claim 18, wherein the radiopaque liquid includes therapeutic drug, wherein the balloon device is a balloon with micro-holes, wherein therapeutic drug in liquid form is delivered from the inside of the balloon through the micro-holes to the treatment site, and wherein ultrasound energy further propagates the drug into and through plaque and into a vessel wall to inhibit restenosis.

20. The device of claim 18, wherein the balloon is coated a drug, and wherein ultrasound energy delivered from the ultrasound emitting member facilitates dispersion of the drug located on the balloon to further propagate the drug into and through plaque and into a vessel wall to inhibits restenosis.

21. The device of claim 19, wherein the ultrasound energy modifies plaque and enhances cellular drug absorption within the vessel wall at the treatment area using one of the following ultrasound waves: longitudinal waves, surface waves, spherical waves, or a combination thereof.

22. A medical device comprising:
a balloon catheter having a guidewire lumen and a balloon;
an ultrasound energy transducer located outside the balloon catheter;
an ultrasound emitting member having a proximal portion attached to the ultrasound energy transducer, and a distal portion extending longitudinally inside the balloon, wherein the ultrasound emitting member is located outside of the guidewire lumen, wherein the ultrasound emitting member delivers ultrasound energy across the balloon when the balloon is inflated, wherein the material of the balloon catheter comprises polymer having acoustic impedance less than 6.0×10⁶ Rayl, and wherein the ultrasound vibrational energy has a frequency range of 1 kHz-1 MHz.

23. The device of claim 22, wherein the balloon has a distal end, and the ultrasound emitting member has a distal end which is positioned inside the balloon in one of the following manners: the distal end of the ultrasound emitting member is freely placed along the length of the balloon, or the distal end of the ultrasound emitting member is attached to the distal end of the balloon.

24. The device of claim 22, wherein the balloon has a distal end, and the ultrasound emitting member has a distal end, wherein the ultrasound emitting member comprises multiple wires, wherein the multiple wires have distal ends, and wherein the multiple wires are positioned inside the balloon in one of the following manners: the distal ends of the multiple wires are freely placed along the length of the balloon, the distal end of the ultrasound emitting member is attached to the distal end of the balloon, or a combination thereof.
